(19) United States
(12) Patent Application Publication
(10) Pub. No.: US 2022/0128251 A1
(43) Pub. Date: Apr. 28, 2022

(54) VENT APPARATUSES AND METHODS OF USING THE SAME

(71) Applicant: Edward Zdunek, Crystal Lake, IL (US)

(72) Inventor: Edward Zdunek, Crystal Lake, IL (US)

(21) Appl. No.: 17/519,174

(22) Filed: Nov. 4, 2021

Related U.S. Application Data
(63) Continuation-in-part of application No. 17/353,040, filed on Jun. 21, 2021, now abandoned.
(60) Provisional application No. 63/041,217, filed on Jun. 19, 2020.

(51) Int. Cl. F24F 8/50; F24F 8/108; F24F 13/08; F24F 7/06; A61L 9/14

(52) U.S. Cl. CPC F24F 8/50; F24F 8/108; F24F 13/085; F24F 7/065; A61L 2209/16; A61L 2209/14; A61L 2209/134; A61L 2209/11; A61L 9/14

(57) ABSTRACT

Vent apparatuses comprise a filter comprising a pleasant scent such that when forced air passes therethrough, the scent on the filter is distributed into the room. Alternatively, a tank and/or reservoir may be disposed adjacent the vent, whereby upon activation a spray of a scent may be introduced into the airstream of the forced air and thereby distributed around the room, or the spray may be forced onto the filter, which may act as a substrate holding the scent thereon such that when forced air passes therethrough. In an alternative embodiment, the vent comprises fan blades that generate electricity from the forced air flowing therethrough for powering electronic devices and/or for charging batteries.

### CROSS-REFERENCE TO RELATED APPLICATIONS

The present invention claims priority as a continuation-in-part application to U.S. patent application Ser. No. 17/353,040, filed Jun. 21, 2021, which claims priority to U.S. Prov. Pat. App. No. 63/041,217, titled “Vent Apparatuses and Methods of Using the Same,” filed Jun. 19, 2020, each of which is incorporated herein by reference in its entirety.

### TECHNICAL FIELD

The present invention relates to vent apparatuses. Specifically, in a first embodiment, a vent apparatus in a room comprises a filter comprising a pleasant scent such that when forced air passes therethrough, the scent on the filter is distributed into the room. Alternatively, a tank and/or reservoir may be disposed adjacent the vent, whereby upon activation a spray of a scent may be introduced into the airstream of the forced air and thereby distributed around the room, or the spray may be forced onto the filter, which may act as a substrate holding the scent thereon such that when forced air passes therethrough, the scent is imparted to the airflow. In an alternative embodiment, the vent comprises fan blades that generate electricity from the forced air flowing therethrough for powering electronic devices and/or for charging batteries.

### BACKGROUND

Most buildings include vents for moving air from one place to another. Indeed, many houses include central HVAC systems where forced air, whether ambient air, heated air, or cooled air, is forced into a room. The forced air can be used simply for circulation of the air within the room, for adjusting the air temperature within a room, and/or for other like purposes.

Oftentimes, it is desirable to also change the scent profile of a room, such as if there are unpleasant odors or if a user simply desires to smell a different scent. There are many ways of accomplishing this, from very low-tech products, such as simply adding flowers or bowls of scented potpourri material, to higher tech products, such as using compressed gas canisters having scented chemicals therein or scent reservoirs that plug into electrical outlets. Scent diffusers are also often used, whereby reservoirs of water and chemical scents are diffused throughout a room via electricity.

Typically, these products that are used to change the scent profile of a room tend to remain localized. In other words, a product used to impart a scent to a room generally stays local to where the product is placed. Put another way, it is often difficult to spread the scent from the location of the product to other areas of the room so that the scent fills the entirety of the room. A need, therefore, exists for an improved product for spreading scent around a room. More specifically, a need exists for a vent apparatus that imparts a scent that is carried around a room via forced air through the vent.

In addition, products that impart scents within rooms are often difficult to control. For example, many simply provide scents at all times, no matter whether necessary. Specifically, low tech products are often difficult to “turn on” or “turn off” when desired, as the scent is typically imparted at all times. Moreover, higher tech products tend to require specific complicated interaction when more scent is desired. For example, diffusers are often simply activated all the time, and when one wants more scent within a room, he or she must add scented chemicals to the diffuser to replenish the same. This may involve opening the reservoir, adding scented chemicals to the reservoir, and ensuring that the diffuser is operating properly. A need, therefore, exists for a vent apparatus that imparts a scent when desired. More specifically, a need exists for a vent apparatus that allows a user to easily control when a scent is imparted to a room.

While vents that have forced air pushed therethrough may effectively move air from one location to another, the forced air typically has energy that can be harvested. Oftentimes, the forced air has plenty of energy to spread throughout a room, thereby fulfilling its primary purpose of either circulating the air within a room or for changing a temperature within the room. However, any left-over energy not required for this purpose is typically lost. A need, therefore, exists for a vent apparatus that can capture left-over energy from forced air through the vent apparatus. More specifically, a need exists for a vent apparatus that may power electrical devices and/or charge batteries utilizing the excess energy.

### SUMMARY OF THE INVENTION

The present invention relates to vent apparatuses. Specifically, in a first embodiment, a vent apparatus in a room comprises a filter comprising a pleasant scent such that when forced air passes therethrough, the scent on the filter is distributed into the room. Alternatively, a tank and/or reservoir may be disposed adjacent the vent, whereby upon activation a spray of a scent may be introduced into the airstream of the forced air and thereby distributed around the room, or the spray may be forced onto the filter, which may act as a substrate holding the scent thereon such that when forced air passes therethrough. In an alternative embodiment, the vent comprises fan blades that generate electricity from the forced air flowing therethrough for powering electronic devices and/or for charging batteries.

To this end, in an embodiment of the present invention, a vent apparatus is provided. The vent apparatus comprises: a vent cover comprising a plurality of openings for air to flow therethrough; a filter for filtering particulate material from air flowing therethrough; a reservoir comprising a chemical scent material; and a dispersion tube configured to disperse the chemical scent from the reservoir.

In an embodiment, the filter comprises a honeycomb construction.

In an embodiment, the vent apparatus further comprises: at least one nozzle on the dispersion tube, wherein the nozzle disperses the chemical scent.

In an embodiment, the at least one nozzle is positioned to disperse the chemical scent onto the filter.

In an embodiment, the at least one nozzle is positioned to disperse the chemical scent into an airflow flowing therethrough.

In an embodiment, the vent apparatus further comprises: a plurality of nozzles interconnected to the reservoir through one or more dispersion tubes, wherein the plurality of nozzles disperses the chemical scent.

In an embodiment, the chemical scent is configured to be dispersed from the reservoir automatically.

In an embodiment, the chemical scent is configured to be dispersed from the reservoir at regular time intervals.

In an embodiment, the vent apparatus further comprises: a lever or button, wherein the lever or button is configured to disperse the chemical scent from the reservoir when pushed.

In an embodiment, the vent apparatus further comprises: a software application on a computing device configured to control the release of the chemical scent, wherein the software application is interconnected with the vent apparatus.

In an alternate embodiment of the present invention, an electricity generating apparatus is provided. The electricity generating device comprises: a vent on a duct within a forced-air HVAC system; a turbine blade system within the duct comprising a plurality of turbine blades, wherein the plurality of turbine blade system is configured to spin when airflow is forced through the duct; and an electrical generator interconnected to the turbine blade system.

In an embodiment, the turbine blade system is positioned within the vent.

In an embodiment, the electricity generating apparatus further comprises: a vent cover, wherein the turbine blade system is positioned adjacent the vent cover.

In an embodiment, the electricity generating apparatus further comprises: a battery interconnected to the generator, wherein the battery is configured to store electricity generated by the generator.

In an embodiment, the electricity generating apparatus further comprises: a plug-in port interconnected with the battery, wherein the plug-in port is configured to accept plugs from electrical devices for transferring power from the battery to the electrical devices.

In an embodiment, the electricity generating apparatus further comprises: a load attached to the battery, wherein the load uses electricity stored in the battery.

In an embodiment, each of the plurality of turbine blades comprises a pocket for catching the airflow forced through the duct.

In an embodiment, the turbine blade system comprises an axial rod extending from the center of the plurality of turbine blades.

In an embodiment, the electricity generating apparatus further comprises: an axial rod extending into the generator, wherein the axial rod comprises an arm disposed at roughly ninety degrees to the axial rod, wherein the arm is connected to a distal end of one of the turbine blades.

In an embodiment, the electricity generating apparatus further comprises: a housing surrounding the turbine blade system, wherein the housing comprises a first plurality of magnets disposed around the inside perimeter of the housing; a second plurality of magnets disposed on the distal ends of the plurality of turbine blades, wherein the first plurality of magnets on the housing and the second plurality of magnets on the turbine blades have like poles facing each other thereby repelling each other.

It is, therefore, an advantage and objective of the present invention to provide an improved product for spreading scent around a room.

More specifically, it is an advantage and objective of the present invention to provide a vent apparatus that imparts a scent that is carried around a room via forced air through the vent.

Further, it is an advantage and objective of the present invention to provide a vent apparatus that imparts a scent when desired.

More specifically, it is an advantage and objective of the present invention to provide a vent apparatus that allows a user to easily control when a scent is imparted to a room.

Still further, it is an advantage and objective of the present invention to provide a vent apparatus that can capture left-over energy from forced air through the vent apparatus.

More specifically, it is an advantage and objective of the present invention to provide a vent apparatus that may power electrical devices and/or charge batteries utilizing the excess energy.

Additional features and advantages of the present invention are described in, and will be apparent from, the detailed description of the presently preferred embodiments and from the drawings.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 illustrates a perspective view of a vent apparatus for filtering air and/or dispersing a chemical scent into an airflow in an embodiment of the present invention.

FIG. 2 illustrates a side view of a vent apparatus in an embodiment of the present invention.

FIG. 3 illustrates a perspective view of a vent apparatus for generating electricity from airflow in an embodiment of the present invention.

FIG. 4 illustrates a perspective view of a vent apparatus for generating electricity from airflow in an embodiment of the present invention.

### DETAILED DESCRIPTION OF THE PRESENTLY PREFERRED EMBODIMENTS

The present invention relates to vent apparatuses. Specifically, in a first embodiment, a vent apparatus in a room comprises a filter comprising a pleasant scent such that when forced air passes therethrough, the scent on the filter is distributed into the room. Alternatively, a tank and/or reservoir may be disposed adjacent the vent, whereby upon activation a spray of a scent may be introduced into the airstream of the forced air and thereby distributed around the room, or the spray may be forced onto the filter, which may act as a substrate holding the scent thereon such that when forced air passes therethrough, the scent is imparted to the airflow. In an alternative embodiment, the vent comprises fan blades that generate electricity from the forced air flowing therethrough for powering electronic devices and/or for charging batteries.

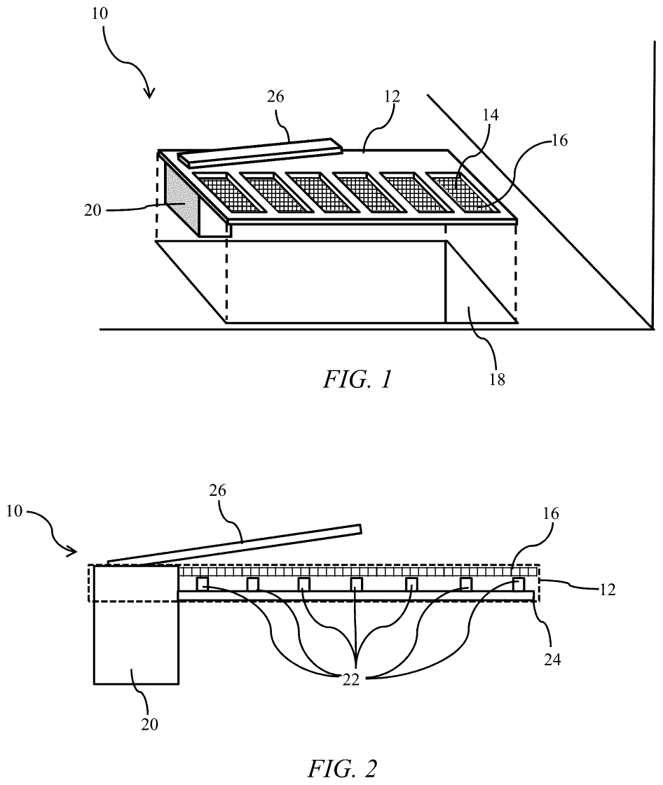

In a first embodiment of the present invention, illustrated in FIG. 1 illustrates a vent apparatus 10 comprising a vent cover 12 having a plurality of openings 14 to allow air to flow therethrough. Below each of the plurality of openings 14 may be a filter 16 that may be configured to filter the air that flows therethrough. The vent apparatus 10 may be positioned within a vent opening 18 that may allow airflow from an HVAC system or other forced air system.

The filter 16 may retain particulates flowing within the airflow to cleanse the air flowing therethrough. The filter may be any filter apparent to one of ordinary skill in the art; however, in a preferred embodiment, the filter may be of a honeycomb construction to allow better airflow than other types of filters while still retaining particulates. The filter may further have a manual release that may allow for easy replacement by a user thereof, such as ball detents and/or magnets, which may allow for the removal of the filter when full, thereby allowing airflow therethrough. An indicator (not shown) may be utilized to communicate to a user that the filter is full.

The filter 16 may be embedded with a scent that may be imparted to airflow flowing therethrough. In a preferred embodiment, illustrates in FIGS. 1 and 2, the vent apparatus 10 may have a reservoir 20 having an amount of a chemical scent contained therein which may be utilized to impart the chemical scent to the apparatus 10 and, preferably, onto the filter 16, thereby imparting the scent to the air flowing therethrough. The reservoir may be refillable or replaceable so that when empty, additional scent may be added, or if a user wishes to change the scent, he or she may simply swap out the chemical scent therein.

The chemical scent contained within the reservoir may be imparted to the airflow through nozzles 22 on a distribution line 24, as illustrated in FIG. 2, which is a side view of the vent apparatus 10. Thus, the chemical scent may flow through distribution line 24 and out through nozzles 22 into the airflow that is flowing through the vent apparatus 10. The nozzles 22 may be positioned in any location, although in a preferred embodiment, the nozzles 22 are positioned in multiple locations evenly to provide even distribution of scent within the airflow.

Preferably, the chemical scent is distributed through the nozzles 22 to the filter to impart the scent onto the filter 16, although it should be noted that the nozzles 22 may simply impart the chemical scent directly to the airflow without a filter. However, by imparting the scent directly to the filter 16, the scent may last longer as it diffuses from the filter medium.

The chemical scent may be imparted through the nozzles 22, or in any other manner from the reservoir 20 manually or automatically. If automatically, the chemical scent may be distributed through the nozzles 22, or in any other manner, at regular time intervals. Thus, the vent apparatus 10 may have mechanical and/or electrical means of distributing the chemical scent. For example, an electronic timer mechanism may activate a pump that may pump the chemical scent through the distribution line 24 and out the nozzles 22.

In an alternate embodiment, the airflow through the vent apparatus may pressurize the reservoir, wherein the pressure of the airflow may automatically force the scent to be distributed through the nozzles 22. Thus, if airflow increases, pressure within the reservoir may increase, which may increase the distribution of the chemical scent through the nozzles 22, thereby aiding in disbursement of the chemical scent therefrom.

Alternatively, the chemical scent may be distributed through a manual process, such as by manually pushing a switch, lever or foot pedal that may trigger the distribution of the scent therefrom. For example, a foot pedal lever 26 may be pushed, preferably by a user's foot, to release chemical scent from the reservoir 20. The lever 26 may mechanically and/or electrically activate a pump that may distribute the chemical scent. The lever 26 may be adjustable so that the same may not be accidentally pressed by a child, animal, another adult, or the like. Specifically, the lever 26 may require a specific amount of force to prevent accidental release of the chemical scent. Other mechanisms may also be utilized, such as switches accessible to a user, such as on a wall within a user's reach, or via a smart device, such as a smartphone that may be tied through a computer network to the vent apparatus, wherein the scent may be imparted via selection on an app on the smart device.

Whether the chemical scent is distributed automatically or manually, as described above, the release rate of the spray may be specifically controlled to optimize the dispersal. For example, the release rate of the spray may be slowed to allow dispersal of the chemical scent into the airflow stream at the desired rate.

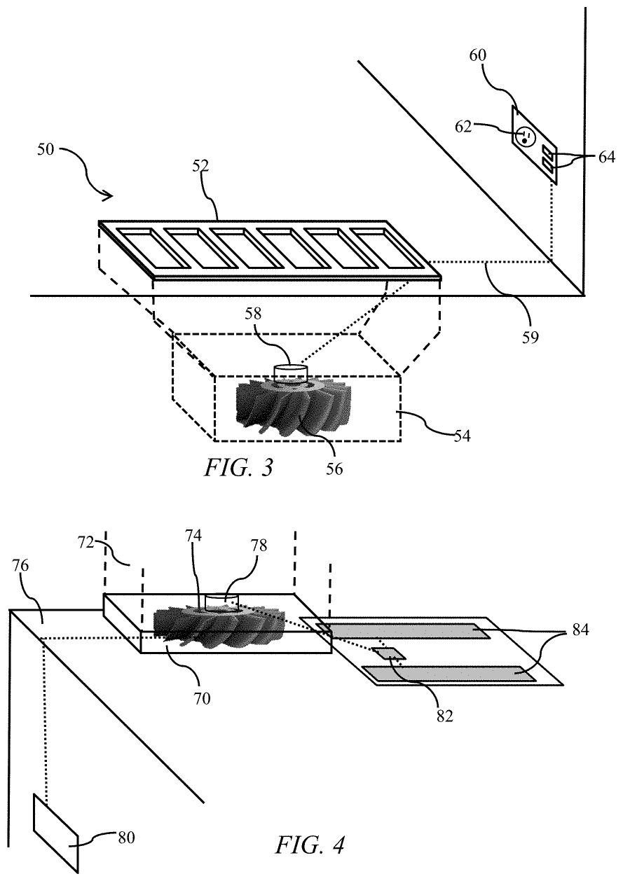

In an alternate embodiment of the present invention, illustrated in FIGS. 3 and 4, a vent apparatus 50 may have a vent cover 52 and a turbine blade apparatus 54 comprising a plurality of turbine blades 56 that may easily turn when air flows therearound and therethrough. The turbine blade apparatus 54 may be disposed beneath the vent cover 52, either vertically or horizontally within the HVAC ductwork so as to interact with the forced air flowing therethrough, although it should be noted that the fan turbine can be placed in any location, such as over a vent cover, if desired, and/or in any orientation apparent to one of ordinary skill in the art. An electrical generator 58 may be interconnected with the fan blades 56 and may be configured to generate electricity as the turbine blades 56 turn from the forced air flowing therethrough.

The electricity generated therefrom may be utilized to power electronic devices, recharge batteries, or may be utilized to allow electrical and/or electronic devices to be powered therefrom, such as through a receptacle 60, distributed thereto via a power cord 59. The receptacle 60 may have ports, such as an electrical outlet 62 and/or USB charging ports 64 for plugging and charging electronic devices via USB. Alternatively, apparatuses may be powered, such as low power electrical lights, such as LED lights, may be powered by the generator 58.

In a related embodiment, illustrated in FIG. 4, a vent apparatus 70 may be positioned within or over a supply and/or exhaust vent system 72 within a ceiling or a wall. The vent apparatus 70 may have a plurality of turbine blades 74 that may turn as forced air flows therethrough. As noted above, the vent apparatus 70 may be positioned on or within a ceiling 76 instead of on a floor, as shown in the other drawings herein.

The turbine blades 74 may be interconnected with an electricity generator 78 that may generate electricity that may supply electrical power to an outlet 80, which may be similar to outlet 60, described above. Alternatively, the electrical generator may supply power to a battery 82 that may supply power to lights 84. For example, the battery 82 may charge and be ready to supply power to lights 84 during an emergency to provide emergency lighting, such as if regular power goes out.

In embodiments of the present invention, the turbine blades 74 may be sized and shaped to concentrate the flow of air thereover, thereby turning the blades more easily. For example, each of the plurality of turbine blades may have a pocket therein that catches air moving therethrough.

Moreover, while the turbine blades may have an axial rod extending from the center of the turbine blades which may extend into the generator for electricity generation, the axial rod may have a ninety-degree arm attached to a distal end of a turbine blade, thereby increasing the torque for the rotation of the axial rod. In another embodiment, the turbine blades may include a plurality of magnets on distal ends thereof. The housing thereof may further have a ring of magnets, such that the magnets of the housing and the magnets on the distal ends of the turbine blades have like poles so that they repel each other. This may increase the speed of the turbine making it more effective, thereby increasing the movement of the air through the system, reducing strain on the electric motor, and saving power by reducing back pressure.

The embodiments described herein may be combined together, such that a vent apparatus comprises a filter and/or a chemical scent reservoir for dispersing chemical scent, and an electrical generator comprising a plurality of turbine blades for generating electricity. In an embodiment, the electricity generated by the turbine blades may be utilized to control the chemical scent dispersion, such as via an automatic and/or electrical manual system.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

I claim:

1. A vent apparatus comprising:
a vent cover comprising a plurality of openings for air to flow therethrough;
a filter for filtering particulate material from air flowing therethrough;
a reservoir comprising a chemical scent material; and
a dispersion tube configured to disperse the chemical scent from the reservoir.

2. The vent apparatus of claim 1 wherein the filter comprises a honeycomb construction.

3. The vent apparatus of claim 1 further comprising:
at least one nozzle on the dispersion tube, wherein the nozzle disperses the chemical scent.

4. The vent apparatus of claim 1 wherein the at least one nozzle is positioned to disperse the chemical scent onto the filter.

5. The vent apparatus of claim 4 wherein the at least one nozzle is positioned to disperse the chemical scent into an airflow flowing therethrough.

6. The vent apparatus of claim 4 further comprising:
a plurality of nozzles interconnected to the reservoir through one or more dispersion tubes, wherein the plurality of nozzles disperses the chemical scent.

7. The vent apparatus of claim 1 wherein the chemical scent is configured to be dispersed from the reservoir automatically.

8. The vent apparatus of claim 7 wherein the chemical scent is configured to be dispersed from the reservoir at regular time intervals.

9. The vent apparatus of claim 1 further comprising:
a lever or button, wherein the lever or button is configured to disperse the chemical scent from the reservoir when pushed.

10. The vent apparatus of claim 1 further comprising:
a software application configured to control the release of the chemical scent, wherein the software application is interconnected with the vent apparatus.

11. An electricity generating apparatus comprising:
a vent on a duct within a forced-air HVAC system;
a turbine blade system within the duct comprising a plurality of turbine blades, wherein the plurality of turbine blade system is configured to spin when airflow is forced through the duct; and
an electrical generator interconnected to the turbine blade system.

12. The electricity generating apparatus of claim 11 wherein the turbine blade system is positioned within the vent.

13. The electricity generating apparatus of claim 12 further comprising:
a vent cover, wherein the turbine blade system is positioned adjacent the vent cover.

14. The electricity generating apparatus of claim 11 further comprising:
a battery interconnected to the generator, wherein the battery is configured to store electricity generated by the generator.

15. The electricity generating apparatus of claim 14 further comprising:
a plug-in port interconnected with the battery, wherein the plug-in port is configured to accept plugs from electrical devices for transferring power from the battery to the electrical devices.

16. The electricity generating apparatus of claim 14 further comprising:
a load attached to the battery, wherein the load uses electricity stored in the battery.

17. The electricity generating apparatus of claim 11 wherein each of the plurality of turbine blades comprises a pocket for catching the airflow forced through the duct.

18. The electricity generating apparatus of claim 11 wherein the turbine blade system comprises an axial rod extending from the center of the plurality of turbine blades.

19. The electricity generating apparatus of claim 11 further comprising:
an axial rod extending into the generator, wherein the axial rod comprises an arm disposed at roughly ninety degrees to the axial rod, wherein the arm is connected to a distal end of one of the turbine blades.

20. The electricity generating apparatus of claim 11 further comprising:
a housing surrounding the turbine blade system, wherein the housing comprises a first plurality of magnets disposed around the inside perimeter of the housing;
a second plurality of magnets disposed on the distal ends of the plurality of turbine blades, wherein the first plurality of magnets on the housing and the second plurality of magnets on the turbine blades have like poles facing each other thereby repelling each other.
